(19) United States
(12) Patent Application Publication
(10) Pub. No.: US 2026/0062674 A1
(43) Pub. Date: Mar. 5, 2026

(54) METHOD FOR IN VITRO STORAGE OF ISOLATED MITOCHONDRIA

(71) Applicant: CHONGQING CHUANYINGXU BIOTECHNOLOGY CO., LTD, CHONGQING (CN)

(72) Inventor: XING ZHOU, CHONGQING (CN)

(21) Appl. No.: 19/378,989

(22) Filed: Nov. 4, 2025

Related U.S. Application Data
(63) Continuation of application No. PCT/CN2024/101513, filed on Jun. 26, 2024.

(30) Foreign Application Priority Data
May 5, 2023 (CN) 202310499889.1

(51) Int. Cl. C12N 5/078; C12N 5/00; C12N 9/76

(52) U.S. Cl. CPC C12N 5/0641; C12N 5/0087; C12N 9/6427; C12Y 304/21004; C12N 2509/10

(57) ABSTRACT

The present invention relates to the technical field of biology, and aims to solve the problems of poor in vitro preservation effect and short preservation time for mitochondria. In order to solve the problems, mitochondria-free cells are innovatively used as carriers to provide storage conditions closest to an intracellular environment for mitochondria, thereby protecting the mitochondrial function and activity. The present invention provides a method for in vitro storage of isolated mitochondria, which mainly includes storing isolated mitochondria within mitochondria-free recipient cells, such that the mitochondrial function and activity are significantly protected and the in vitro preservation time of mitochondria is greatly prolonged. Furthermore, the release of mitochondria contained in red blood cells is facilitated without complex separation steps and reagents, and the operability of such a storage system is enhanced.

### BACKGROUND OF THE INVENTION

### 1. Technical Field

The present invention relates to the technical field of biology, and in particular, relates to a method for in vitro storage of isolated mitochondria.

### 2. Description of Related Art

Mitochondria are involved in the occurrence and development of various pathophysiological processes, making mitochondrial research a focus that has increasingly attracted the attention of researchers. Maintaining the in vitro activity and function of mitochondria is essential for carrying out the mitochondrial research.

In the current clinical and experimental settings, isolated mitochondria are mainly preserved in a mitochondrial storage solution or 0.01 M phosphate-buffered saline (PBS) buffer solution, both of which primarily maintain the mitochondrial activity by providing an appropriate osmotic pressure and a neutral pH environment for the isolated mitochondria. However, these solutions mainly consist of inorganic salts and lack antioxidant substances, showing limited protective effect on the activities of critical enzymes in the mitochondria. As a result, they cannot offer long-term in vitro storage for mitochondria, leading to restriction of the related research and application of mitochondria.

With existing techniques, mitochondria can be introduced into nucleated cells containing intact organelle units, such as white blood cells and lymphocytes. These techniques typically involve introducing mitochondria into cells that have a complete structure but impaired functions, with the primary purpose of repairing the impaired mitochondrial functions of recipient cells through mitochondrial transplantation to ultimately restore or enhance the functions of the recipient cells, instead of the purpose of preserving mitochondria in vitro. Moreover, mitochondria perform biosynthetic and energy-supplying functions within target cells, and their respiratory patterns are regulated by the environment of the target cells, leading to reprogrammed functions and types of the mitochondria in the recipient cells. This causes the mitochondria to lose their original characteristics. Such mitochondria preservation based on cellular transplantation fails to maintain and protect the original characteristics of the mitochondria.

### BRIEF SUMMARY OF THE INVENTION

In view of the problems of poor in vitro preservation effect and short preservation time for mitochondria, the present invention innovatively utilizes mitochondria-free cells as carriers to provide storage conditions closest to an intracellular environment for mitochondria to protect the mitochondrial function and activity, in order to solve the above-mentioned problems.

The inventors have found that for nucleated cells, heterogeneous mitochondria are eventually cleared by recipient cells when the cells'own mitochondria are not cleared, making it impossible to preserve the exogenous mitochondria for a long time. Meanwhile, the presence of complex cellular components and organelles in these nucleated cells complicates the re-extraction of mitochondria and thus reduces the purity of mitochondria.

Specifically, the inventors utilize mitochondria-free cells as carriers (preferably, mature red blood cells and other cells that naturally lack nuclei and organelles) to provide storage conditions closest to an intracellular environment for mitochondria. In this way, the mitochondrial function and activity are significantly protected, and the in vitro preservation time of mitochondria is prolonged. Meanwhile, thanks to the simple composition and structure of red blood cells, mitochondria as sensitive organelles are less susceptible to respirator pattern reprogramming triggered by intracellular components. Moreover, the mitochondria in red blood cells are easy to re-collect, that is, the mitochondria contained in the red blood cells is convenient to release, without complex separation steps and reagents, thereby increasing the operability of such a storage system.

The embodiments of the present invention are implemented by technical solutions as follows:
a method for in vitro storage of isolated mitochondria mainly includes storing the isolated mitochondria in mitochondria-free recipient cells; preferably, the recipient cells are anucleate, which is more conducive to the entry of exogenous organelles; and specifically, the method includes the steps of:
S1: extracting red blood cells and mitochondria from the same or different subjects, respectively, and preparing a red blood cell suspension and a mitochondria suspension, respectively; and
S2: introducing the mitochondria into the red blood cells: mixing the red blood cell suspension and the mitochondria suspension from S1, centrifuging a resulting mixture to remove a supernatant to obtain a mitochondria-containing red blood cell pellet, and subsequently dispersing the mitochondria-containing red blood cell pellet in a red blood cell preservation solution.

More further, the method includes the steps as follows.

### 1.1 Extracting Red Blood Cells

Mice (nude, Balb/C, C57BL/6, or Kunming mice) are anesthetized by intraperitoneal injection of 1-3 mL of 4%-10% anesthesia (phenobarbital sodium, pentobarbital sodium, pentothal sodium, ethyl carbamate, or chloral hydrate); 0.1-1.5 mL of peripheral blood is collected from the mice, and centrifuged for 1-20 min at 100-2000 g and 4° C.-25° C.; and after centrifugation, a supernatant is removed to obtain a red blood cell pellet, which is then resuspended in 1-1000 mL of PBS for later use.

### 1.2 Extracting Mitochondria

The mice (nude, Balb/C, or C57BL/6) are sacrificed by cervical dislocation; and then, the tissues (from heart, muscle, spleen, kidney or liver) or peripheral white blood cells of the mice are isolated. The tissues are then digested for 15-120 min with trypsin (0.05%-0.5%) at 4° C.-37° C. After mechanical grinding, the tissues are filtered through a 50-200-mesh cell strainer to obtain a single-cell suspension, which is then centrifuged for 1-20 min at 100-2000 g and 4° C.-25° C.; and after centrifugation, a supernatant is removed to obtain a cell pellet. The cell pellet is treated using a mitochondria isolation buffer (1-100 times the volume of the cell pellet) and a mitochondrial lysis buffer for 5-120 min at 4° C.-25° C., followed by centrifuging for 1-20 min at 100-2000 g and 4° C.-25° C.; and after centrifugation, a supernatant is removed to obtain a mitochondria pellet, which is then resuspended in 1-1000 mL of PBS for later use.

### 1.3 Introducing the Mitochondria Into the Red Blood Cells

The mitochondria suspension from 1.2 and the red blood cell suspension from 1.1 are mixed at a ratio of 20:1 to 1:20; a resulting mixture is centrifuged for 1-20 min at 100-2000 g and 4° C.-25° C.; and after centrifugation, a supernatant is removed to obtain a mitochondria-containing red blood cell pellet, which is then dispersed in 1-100 mL of a red blood cell preservation solution.

The technical solutions of the embodiments of the present invention have at least the advantages and beneficial effects as follows.

In view of the problems of poor in vitro preservation effect and short preservation time for mitochondria, the present invention innovatively utilizes cells naturally lacking nuclei and organelles, preferably mature red blood cells, as carriers to provide storage conditions closest to an intracellular environment for mitochondria. In this way, the mitochondrial function and activity are significantly protected, and the in vitro preservation time for mitochondria is prolonged. Meanwhile, thanks to the simple composition and structure of red blood cells, mitochondria as sensitive organelles are less susceptible to respirator pattern reprogramming triggered by intracellular components. Moreover, the mitochondria in red blood cells are easy to re-collect, that is, the mitochondria contained in the red blood cells are more convenient to release, without complex separation steps and reagents, thereby increasing the operability of such a storage system.

### BRIEF DESCRIPTION OF THE SEVERAL VIEWS OF THE DRAWINGS

To illustrate the technical solutions of the embodiments of the present invention more clearly, the following briefly introduces the accompanying drawings required in the embodiments. It should be understood that the accompanying drawings below only show some embodiments of the present invention, and thus should not be construed as limiting the scope. Those of ordinary skill in the art may still derive other drawings from these accompanying drawings without creative efforts.

FIG. 1 shows schematic images of co-localization of red blood cells and mitochondria according to Experimental Example 1 of the present invention (Mito Tracker: mitochondria label; DiO: cell membrane label);

FIG. 2 shows a schematic diagram of encapsulation efficiency at different mitochondria/red blood cell mixing ratios according to Experimental Example 2 of the present invention;

FIG. 3 shows a schematic diagram of protective effects of red blood cells on mitochondrial activity according to Experimental Example 3 of the present invention; and

FIG. 4 shows a schematic diagram of repairing effects of mitochondria from different storage methods for injured myocardial cells according to Experimental Example 4 of the present invention.

### DETAILED DESCRIPTION OF THE INVENTION

For clearer description of the objects, technical solutions, and advantages of the embodiments of the present invention, the technical solutions in the embodiments of the present invention will be described clearly and completely below. Embodiments without clear indication of specific conditions are carried out following the conventional conditions or the conditions recommended by the manufacturers. The reagents or instruments used without clear indication of manufacturers are all commercially available conventional products.

### Example 1

A method for in vitro storage of isolated mitochondria included steps as follows.

C57BL/6 mice were anesthetized by intraperitoneal injection of 1.5 mL of 4% chloral hydrate; 0.5 mL of peripheral blood was collected from the mice, and centrifuged for 10 min at 800 g and 4° C.; and after centrifugation, a supernatant was removed to obtain a red blood cell pellet.

Then, the heart tissues of the mice were isolated, and digested for 60 min with 0.25% trypsin at 37° C. After mechanical grinding, the tissues were filtered through a 70-mesh cell strainer to obtain a single-cell suspension, which was then centrifuged for 20 min at 300 g and 25° C., and after centrifugation, a supernatant was removed to obtain a cell pellet, which was then resuspended in 1 mL of PBS for later use; the cell pellet was treated using a mitochondria isolation buffer (10 times the volume of the cell pellet) and a mitochondrial lysis buffer for 10 min at 4° C., followed by centrifuging for 5 min at 800 g and 25° C., and after centrifugation, a supernatant was removed to obtain a mitochondria pellet, which was then resuspended in 1 mL of PBS for later use; and the mitochondria suspension and the red blood cell suspension were mixed at a ratio of 1:1, a resulting mixture was centrifuged for 20 min at 800 g and 4° C., and after centrifugation, a supernatant was removed to obtain a mitochondria-containing red blood cell pellet, which was then dispersed in 1 mL of PBS as a red blood cell preservation solution.

### Example 2

Kunming mice were anesthetized by intraperitoneal injection of 1.5 mL of 5% phenobarbital sodium; 1.5 mL of peripheral blood was collected from the mice, and centrifuged for 5 min at 800 g and 25° C.; and after centrifugation, a supernatant was removed to obtain a red blood cell pellet. Then, the heart tissues of the mice were isolated, and digested for 120 min with 0.20% trypsin at 25° C. After mechanical grinding, the tissues were filtered through a 40-mesh cell strainer to obtain a single-cell suspension, which was then centrifuged for 15 min at 800 g and 25° C., and after centrifugation, a supernatant was removed to obtain a cell pellet, which was then resuspended in 2 mL of PBS for later use; the cell pellet was treated using a mitochondria isolation buffer (10 times the volume of the cell pellet) and a mitochondrial lysis buffer for 5 min at 25° C., followed by centrifuging for 20 min at 800 g and 25° C., and after centrifugation, a supernatant was removed to obtain a mitochondria pellet, which was then resuspended in 5 mL of PBS for later use; and the mitochondria suspension and the red blood cell suspension were mixed at a ratio of 2:1, a resulting mixture was centrifuged for 20 min at 800 g and 25° C., and after centrifugation, a supernatant was removed to obtain a mitochondria-containing red blood cell pellet, which was then dispersed in 2 mL of PBS as a red blood cell preservation solution.

### Example 3

Balb/C mice were anesthetized by intraperitoneal injection of 1 mL of 8% chloral hydrate; 1 mL of peripheral blood was collected from the mice, and centrifuged for 10 min at 800 g and 4° C.; and after centrifugation, a supernatant was removed to obtain a red blood cell pellet. Then, the heart tissues of the mice were isolated, and digested for 60 min with 0.5% trypsin at 37° C. After mechanical grinding, the tissues were filtered through a 70-mesh cell strainer to obtain a single-cell suspension, which was then centrifuged for 20 min at 400 g and 4° C., and after centrifugation, a supernatant was removed to obtain a cell pellet, which was then resuspended in 1 mL of PBS for later use; the cell pellet was treated using a mitochondria isolation buffer (10 times the volume of the cell pellet) and a mitochondrial lysis buffer for 4 min at 25° C., followed by centrifuging for 20 min at 600 g and 25° C., and after centrifugation, a supernatant was removed to obtain a mitochondria pellet, which was then resuspended in 10 mL of PBS for later use; and the mitochondria suspension and the red blood cell suspension were mixed at a ratio of 1:5, a resulting mixture was centrifuged for 5 min at 800 g and 20° C., and after centrifugation, a supernatant was removed to obtain a mitochondria-containing red blood cell pellet, which was then dispersed in 5 mL of PBS as a red blood cell preservation solution.

### Example 4

C57BL/6 mice were anesthetized by intraperitoneal injection of 0.5 mL of 10% pentobarbital sodium; 0.5 mL of peripheral blood was collected from the mice, and centrifuged for 10 min at 800 g and 4° C.; and after centrifugation, a supernatant was removed to obtain a red blood cell pellet. Then, the heart tissues of the mice were isolated, and digested for 60 min with 0.25% trypsin at 37° C. After mechanical grinding, the tissues were filtered through a 700-mesh cell strainer to obtain a single-cell suspension, which was then centrifuged for 20 min at 300 g and 25° C., and after centrifugation, a supernatant was removed to obtain a cell pellet, which was then resuspended in 1 mL of PBS for later use; the cell pellet was treated using a mitochondria isolation buffer (10 times the volume of the cell pellet) and a mitochondrial lysis buffer for 20 min at 4° C., followed by centrifuging for 25 min at 400 g and 25° C., and after centrifugation, a supernatant was removed to obtain a mitochondria pellet, which was then resuspended in 10 mL of PBS for later use; and the mitochondria suspension and the red blood cell suspension were mixed at a ratio of 5:1, a resulting mixture was centrifuged for 20 min at 800 g and 4° C., and after centrifugation, a supernatant was removed to obtain a mitochondria-containing red blood cell pellet, which was then dispersed in 1 mL of saline as red blood cell preservation solution.

### Experimental Example 1—Construction and Verification of Red Blood Cell Storage System for Mitochondria

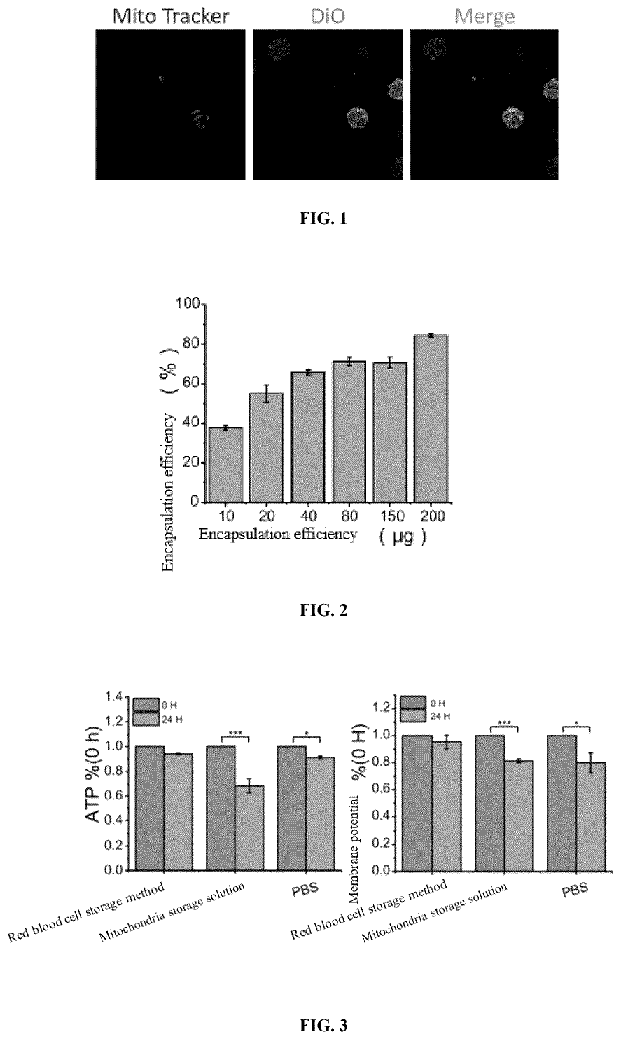

The membranes of red blood cells were labeled with green fluorescent probes DiO, the mitochondria were labeled with Mito Tracker® Red CMX Ros, and then, a labeled mitochondria suspension and a labeled red blood cell suspension were mixed at a ratio of protein content:cell count=80 μg:10×10⁶, respectively. A resulting mixture was centrifuged for 20 min at 800 g and 25° C. After centrifugation, a supernatant was removed to obtain a mitochondria-containing red blood cell pellet, which was then dispersed in 2 mL of a red blood cell preservation solution. After completing the establishment of the storage system, a small amount of sample was carefully pipetted onto a microscope slide, and then, the mitochondria and red blood cells was analyzed for co-localization under a fluorescence confocal microscope. The results showed a high degree of co-localization between the fluorescence of the mitochondria label and the red blood cell membrane label, indicating successful introduction of the mitochondria into the red blood cells by the method described in the present invention. The experimental results were shown in FIG. 1.

### Experimental Example 2—Encapsulation Efficiency of Red Blood Cell Storage System for Mitochondria

The mitochondria were labeled with Mito Tracker® Red CMX Ros, and then, 10 μg, 20 μg, 40 μg, 80 μg, 150 μg or 200 μg of a labeled mitochondria suspension was mixed with 10×10⁶ red blood cells. A resulting mixture was centrifuged for 20 min at 800 g and 25° C. After centrifugation, a supernatant was removed to obtain a mitochondria-containing red blood cell pellet, which was then dispersed in 2 mL of a red blood cell preservation solution. After completing the establishment of storage systems of different mixing ratios, statistical analysis of the fluorescence intensity of the mitochondria label in the red blood cells was conducted using flow cytometry. The results revealed that the highest mitochondrial encapsulation efficiency was achieved by mixing the mitochondria suspension and the red blood cell suspension at a ratio of protein content:cell count=200 μg:10×10⁶. The experimental results were shown in FIG. 2.

### Experimental Example 3

### Protective Effects of Red Blood Cells on Mitochondrial Activity

Mitochondria were stored in red blood cells, PBS, or a commercial mitochondrial storage solution, and then, the APT synthesis capacity and the membrane potential levels of the mitochondria were compared among different storage methods after 24 hours of in vitro storage at 4° C. The results revealed that compared to the freshly isolated mitochondria (stored only in a preservation solution for transfer storage), the mitochondria stored in the red blood cells showed a smaller decrease in ATP content, while the mitochondria stored in the mitochondrial storage solution showed a significant decrease in ATP content; and the mitochondria stored in the red blood cells showed a significantly lower decrease in mitochondrial membrane potential than the other two storage methods. In summary, encapsulating with red blood cells provides a better protective effect for the mitochondrial activity. The experimental results were shown in FIG. 3.

### Experimental Example 4—Repairing Effects of Mitochondria From Different Storage Methods for Injured Myocardial Cells

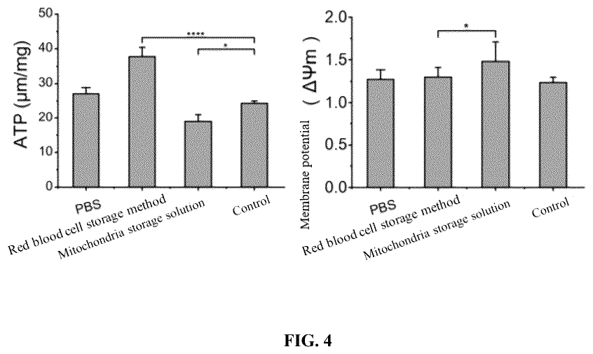

After the establishment of an isoproterenol (ISO)-induced myocardial cell injury model, a transplantation therapy was carried out on myocardial cells by means of mitochondria stored in vitro for 24 hours at 4° C. using different storage methods. The results revealed that the myocardial cells subjected to the transplantation therapy using the mitochondria stored in red blood cells exhibited higher ATP content and membrane potential, indicating that the mitochondria stored in red blood cells still retained potential for the transplantation therapy. The experimental results were shown in FIG. 4.

In summary, the present invention provides storage conditions closest to an intracellular environment for mitochondria by utilizing red blood cells free of mitochondria and nuclei as carriers, such that the mitochondrial function and activity are significantly protected, and the in vitro preservation time of mitochondria is prolonged. Meanwhile, the nuclei-regulated reprogramming of mitochondrial function and the elimination of heterogeneous mitochondria are avoided. The present invention provides a stable means for in vitro preservation of mitochondria.

In addition, both PBS and the mitochondria storage solution offer only limited protective effect on the mitochondrial activity, by which the mitochondrial activity is significantly reduced after 24 hours of in vitro storage. In contrast, the mitochondria stored in red blood cells at (4±2)° C. can be preserved in vitro for 35-42 days, offering a long-term protective environment for mitochondria and prolonging the in vitro preservation time for mitochondria.

Described above are merely preferred embodiments of the present invention, which are not intended to limit the present invention. For those skilled in the art, various changes and variations can be made to the present invention. Any modification, equivalent substitution, improvement and the like made within the spirit and principle of the present invention shall be construed as being included within the protection scope of the present invention.

What is claimed is:

1. A method for in vitro storage of isolated mitochondria, comprising storing the isolated mitochondria in mitochondria-free recipient cells, wherein the recipient cells are anucleate mature red blood cells in blood; the isolated mitochondria are fully functional mitochondria; and
specifically, the method comprises the steps of:
S1: extracting red blood cells and mitochondria from the same or different subjects, respectively, and preparing a red blood cell suspension and a mitochondria suspension, respectively; and
S2: introducing the mitochondria into the red blood cells: mixing the red blood cell suspension and the mitochondria suspension from S1 at a ratio of 20:1 to 1:20, centrifuging a resulting mixture to remove a supernatant to obtain a mitochondria-containing red blood cell pellet, and then, dispersing the mitochondria-containing red blood cell pellet in a red blood cell preservation solution.

2. The method for in vitro storage of isolated mitochondria according to claim 1, wherein the isolated mitochondria are isolated from donor cells, and the donor cells and the recipient cells are derived from the same or different subjects.

3. The method for in vitro storage of isolated mitochondria according to claim 1, wherein step S1 comprises S11, extracting the red blood cells, which comprises: collecting peripheral blood from a subject, centrifuging the peripheral blood for a period of time, removing a supernatant after the centrifuging to obtain a red blood cell pellet, and resuspending the red blood pellet to obtain a red blood cell suspension.

4. The method for in vitro storage of isolated mitochondria according to claim 1, wherein step S1 further comprises S12, extracting the mitochondria, which comprises: collecting a tissue from a subject, digesting the tissue with trypsin for a period of time, and grinding, sieving and filtering the tissue to prepare a single-cell suspension; and centrifuging to separate the single-cell suspension to obtain a cell pellet, treating the cell pellet, subsequently centrifuging to separate the cell pellet to obtain a mitochondrial pellet, and resuspending the mitochondrial pellet to obtain a mitochondria suspension.

5. The method for in vitro storage of isolated mitochondria according to claim 1, wherein step S1 further comprises S13, extracting the mitochondria, which comprises: collecting peripheral blood from a subject, and removing red blood cells from the peripheral blood using a red blood cell lysis buffer; and centrifuging to separate the resulting peripheral blood to obtain a cell pellet, treating the cell pellet, subsequently centrifuging to separate the cell pellet to obtain a mitochondrial pellet, and resuspending the mitochondrial pellet to obtain a mitochondria suspension.
